(19) United States
(12) Patent Application Publication
(10) Pub. No.: US 2005/0283076 A1
(43) Pub. Date: Dec. 22, 2005

(54) Non-invasive diagnosis of breast cancer using real-time ultrasound strain imaging

(76) Inventors: Nicholas J. Hangiandreou, Rochester, MN (US); Michelle R. Nordland, Pine Island, MN (US); Gina K. Hesley, Rochester, MN (US); Marilyn J. Morton, Rochester, MN (US); J. William Charboneau, Rochester, MN (US); Duane D. Meixner, Lake City, MN (US)

Correspondence Address: QUARLES & BRADY LLP, 411 E. WISCONSIN AVENUE, SUITE 2040, MILWAUKEE, WI, 53202-4497, US

(21) Appl. No.: 11/154,838

(22) Filed: Jun. 16, 2005

Related U.S. Application Data
(60) Provisional application No. 60/581,137, filed on Jun. 18, 2004.

(51) Int. Cl. A61B 8/00

(52) U.S. Cl. 600/443; 600/449

(57) ABSTRACT

A series of ultrasound strain images of a breast lesion are acquired along with corresponding B-mode images using a real-time ultrasound strain imaging system and a free-hand technique. A visual assessment of the lesion is made by the sonographer after image acquisition. A conspicuity metric is calculated from the strain images based on the weighted sum of lesion contrast values in each strain image. The weighting of each lesion contrast value is based on observed characteristics of malignant lesions in a series of strain images. Diagnosis is made based on the visual assessment and the conspicuity metric

### CROSS-REFERENCE TO RELATED APPLICATIONS

This application claims the benefit of U.S. Provisional patent application Ser. No. 60/581,137 filed on Jun. 18, 2004 and entitled “VISUAL AND COMPUTER-AIDED ANALYSIS OF REAL-TIME ULTRASOUND STRAIN IMAGE SEQUENCE FOR DIFFERENTIATION OF BREAST LESIONS”.

### BACKGROUND OF THE INVENTION

Breast cancer is the most common form of cancer in women in the United States and worldwide. According to the World Health Organization (WHO) more than 1.2 million women were diagnosed with breast cancer in 2000. The American Cancer Society (ACS) estimated that 215,000 cases of invasive breast cancer would be diagnosed in the United States and over 40,000 women would die of this disease in 2004. Mammography and clinical breast examination are used to screen for breast cancer. Because both physical findings and mammographic findings of benign and malignant breast abnormalities overlap, screening for breast cancer results in the detection of many benign breast abnormalities that require biopsy for definitive diagnosis. Benign breast biopsies add significant economic costs to mammographic screening as well as emotional and psychological costs of stress and anxiety experienced by women who fear they may have breast cancer. Over one million breast biopsies are performed in the United States each year but less than one-half will result in a diagnosis of breast cancer. The use of specific sonographic features to differentiate benign and malignant breast masses has been evaluated as a means to reduce the number of biopsies performed for benign solid lesions. Although the use of such classification schemes holds potential for the accurate diagnosis of breast masses, most radiologists recommend biopsy of a solid mass to avoid misdiagnosis.

The observation that benign and malignant breast lesions have an inherently different firmness has long been used by clinicians during palpation of the breast: harder and less mobile lesions are considered more likely malignant. Palpation is limited however by lesion size, depth of the lesion, and the background tissue firmness. Methods to image the strain distribution in tissues may overcome these limitations and allow quantification of this qualitative observation. Several different methods, including ultrasound (US) strain imaging, have been developed to measure the relative stiffness of lesions in contrast to the tissue around them

Work by Garra et al “Elastography of breast lesions: initial clinical results”, Radiology 1997; 202:79-86, and other investigators has shown that breast lesion size discrepancies between B-mode ultrasound images and strain images may be a promising way to distinguish benign from malignant lesions (strain imaging lesion-size comparison technique). They found that malignant lesions tend to appear larger on strain images than the corresponding B-mode image. This most likely occurs because of the surrounding desmoplastic reaction which accompanies most malignancies.

Current methods utilizing this observation to predict lesion status from a sequence of strain images require several steps. The first involves imaging the patient and acquiring a set of data that are reconstructed into sequences of B-mode and corresponding strain images. Next an operator must review the image sequences and select B-mode and strain images for lesion segmentation. Manual segmentation of the lesions in both images is performed by tracing the observed lesions borders. Finally, a software program is used to calculate the lesion areas in each of the two images and, the ratio of the strain image area to the B-mode image area, and to compare this area ratio to a previously defined threshold. Area ratios exceeding the threshold are judged to indicate a malignancy, and ratios below the threshold indicate a benign finding. The main limitations of this technique include low specificity for some observers and marked inter-observer variation (mainly in lesion size measurement). Also, the extensive time required to choose the optimal image frame from the cine-loop sequence and to make the lesion size measurements make the routine application of this technique in a typical busy clinical breast imaging practice difficult.

### SUMMARY OF THE INVENTION

The present invention is a method for acquiring and examining ultrasound images of a lesion and diagnosing whether the lesion is malignant or benign. More specifically, the invention includes acquiring a series of ultrasound strain images of a subject lesion and calculating a conspicuity metric based on the weighted sum of lesion contrast values calculated for each strain image in the series. The particular weighting of each lesion contrast value is based on observed characteristics of strain images that render malignant lesions more conspicuous in the series of strain images.

We hypothesize that it is possible to distinguish benign from malignant breast lesions by visually assessing the entire strain image sequence during acquisition. Observed factors such as “ease” of strain image acquisition and clear presentation of the lesion throughout the sequence are evidence of malignancy. This overall impression has been dubbed “conspicuity”. Lesions judged to be more conspicuous during acquisition are predicted to be malignant, while those judged as less conspicuous are predicted to be benign.

The prediction that malignant lesions should be “easy to scan” and appear in a very conspicuous manner throughout the strain sequence is consistent with the physical characteristics of malignant lesions. These lesions are expected to be much firmer, or harder, than the normal breast tissues in which they are embedded giving rise to high strain image contrast. Also, since these lesions are generally well-fixed in the normal tissue matrix and relatively immobile, it is easier for the examining technologist to apply consistent, axial compression and decompression, which produces many frames in the strain sequence which demonstrate the lesion. Benign lesions on the other hand are generally not as firm as malignant lesions, and are relatively mobile and freer to move within the normal tissue matrix. Images of these lesions show less contrast in the strain image sequence than that seen with malignancies. Also, the mobility of these lesions makes it more difficult for the technologist to apply consistent axial compression, since the lesions have a tendency to also move in the lateral and elevational directions. These non-axial motions can cause general failures of the motion tracking algorithm, and strain images that show mainly decorrelation noise and very little if any anatomical structure. Noisy strain image frames do appear in strain sequences of both benign and malignant lesions, but they appear with greater frequency when benign lesions are imaged.

A general object of the invention is to provide an ultrasound method which facilitates the non-invasive diagnosis of a breast lesion. Strain images may be acquired using a freehand method of applying stress to the tissues. This system requires no additional equipment attached to the ultrasound transducer, such as force measurement or tissue loading apparatus, and thus remains relatively robust and simple to operate. The strain imaging capability may be added to a standard clinical ultrasound platform as a software upgrade, with no additional hardware costs.

### BRIEF DESCRIPTION OF THE DRAWINGS

FIG. 1 is a block diagram of an ultrasonic imaging system used to practice the present invention;

FIG. 2 is a pictorial representation of the manner in which the system of FIG. 1 is used to acquire image data;

FIG. 3 is an electrical schematic drawing of a receiver which forms part of the imaging system of FIG. 1;

FIG. 4 is a flow chart of the preferred method for practicing the present invention; and

FIG. 5 is a pictorial representation of a strain image produced in accordance with the method of FIG. 4.

### DETAILED DESCRIPTION OF THE PREFERRED EMBODIMENT

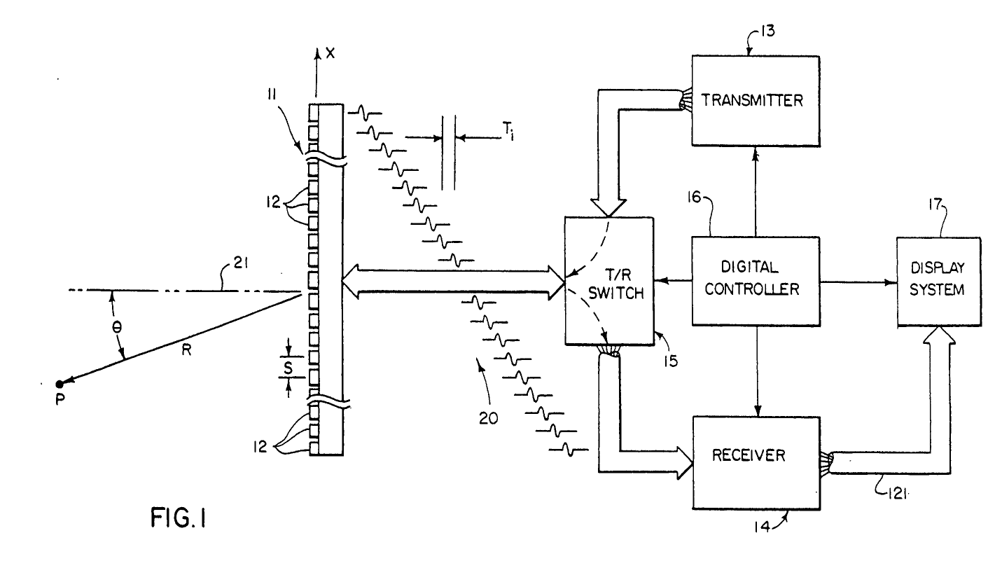

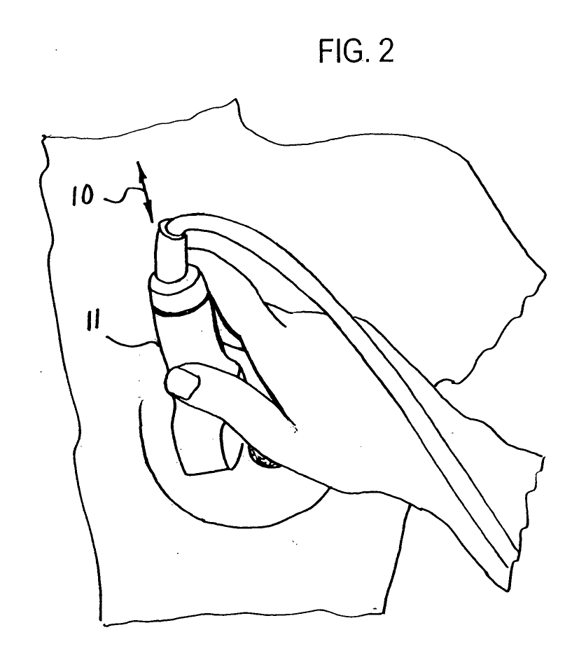

The present invention is presently implemented using a real-time, freehand strain imaging method on a commercially available ultrasound system (Elegra scanner and 7.5L40 linear array transducer 11 sold by Siemens Medical Solutions, Ultrasound Division) and depicted in FIG. 1. The ultrasound imaging is performed at 7.2 MHz by a sonographer while applying freehand, periodic, gentle axial, loading and unloading to the tissues of interest with the transducer 11 as depicted by the arrow 10 in FIG. 2.

Referring particularly to FIG. 1, the ultrasonic imaging system includes a transducer array 11 comprised of a plurality of separately driven piezoelectric elements 12 which each produce a burst of ultrasonic energy when energized by a pulse produced by a transmitter 13. The ultrasonic energy reflected back to the transducer array 11 from the subject under study is converted to an electrical signal by each transducer element 12 and applied separately to a receiver 14 through a set of switches 15. The transmitter 13, receiver 14 and the switches 15 are operated under the control of a digital controller 16 responsive to the commands input by the human operator. A complete scan is performed by acquiring a series of echoes in which the switches 15 are set to their transmit position, the transmitter 13 is gated on momentarily to energize each transducer element 12, the switches 15 are then set to their receive position, and the subsequent echo signals produced by each transducer element 12 are applied to the receiver 14. The separate echo signals from each transducer element 12 are combined in the receiver 14 to produce a single echo signal which is employed to produce a line in an image on a display system 17.

The transmitter 13 drives the transducer array 11 such that the ultrasonic energy produced is directed, or steered, in a beam. A B-mode scan can therefore be performed either by moving the point of origin of this beam from point-to-point along the transducer face, or by steering the beam along different angles rather than physically moving the transducer array 11. To accomplish this in one embodiment the transmitter 13 imparts a time delay (Tᵢ) to the respective pulses 20 that are applied to successive transducer elements 12 to “steer” the ultrasonic beam along different angles. If the time delay is zero (Tᵢ=0), all the transducer elements 12 are energized simultaneously and the resulting ultrasonic beam is directed along an axis 21 normal to the transducer face and originating from the center of the transducer array 11. As the time delay (Tᵢ) is increased as illustrated in FIG. 1, the ultrasonic beam is steered downward from the central axis 21 by an angle θ. A sector scan is performed by progressively changing the time delays Tᵢ in successive excitations. The angle θ is thus changed in increments to steer the transmitted beam in a succession of directions. In the alternative, the ultrasonic beam may be produced by a subset of the transducer elements 12, and rather than being steered at different angles, the beam extends perpendicular from the transducer 11 and moves linearly from one end of the transducer to the other by incrementally moving the subset of active elements 12 along the face of the transducer 11 after each echo signal is acquired.

Referring still to FIG. 1, the echo signals produced by each burst of ultrasonic energy emanate from reflecting objects located at successive positions (R) along the ultrasonic beam. These are sensed separately by each segment 12 of the transducer array 11 and a sample of the magnitude of the echo signal at a particular point in time represents the amount of reflection occurring at a specific range (R). Due to the differences in the propagation paths between an echo origination point P and each transducer element 12, however, these echo signals will not occur simultaneously and their amplitudes will not be equal. The function of the receiver 14 is to amplify and demodulate these separate echo signals, impart the proper time delay to each and sum them together to provide a single echo signal which accurately indicates the total ultrasonic energy reflected from each point P located at range R along the ultrasonic beam.

To simultaneously sum the electrical signals produced by the echoes from each transducer element 12, time delays are introduced into each separate transducer element channel of the receiver 14. The delay introduced in each channel may be divided into two components, one component is referred to as a beam steering time delay, and the other component is referred to as a beam focusing time delay. The beam steering and beam focusing time delays for reception are precisely the same delays (Tᵢ) as the transmission delays described above for the beam steering embodiment. However, the focusing time delay component introduced into each receiver channel is continuously changing during reception of the echo to provide dynamic focusing of the received beam at the range R from which the echo signal emanates.

Under the direction of the digital controller 16, the receiver 14 provides delays during the scan such that the receiver 14 tracks with the particular beam direction or beam location produced by the transmitter 13 and it samples the echo signals at a succession of ranges and provides the proper delays to dynamically focus at points P along the beam. Thus, each emission of an ultrasonic pulse results in the acquisition of a series of data points which represent the amount of reflected sound from a corresponding series of points P located along the ultrasonic beam.

The display system 17 receives the series of data points produced by the receiver 14 and converts the data to a form producing the desired image. For a B-scan, each data point in the series is used to control the brightness of a pixel in the image, and a scan comprised of a series of measurements at successive ultrasound beam lines is performed to provide the data necessary for display.

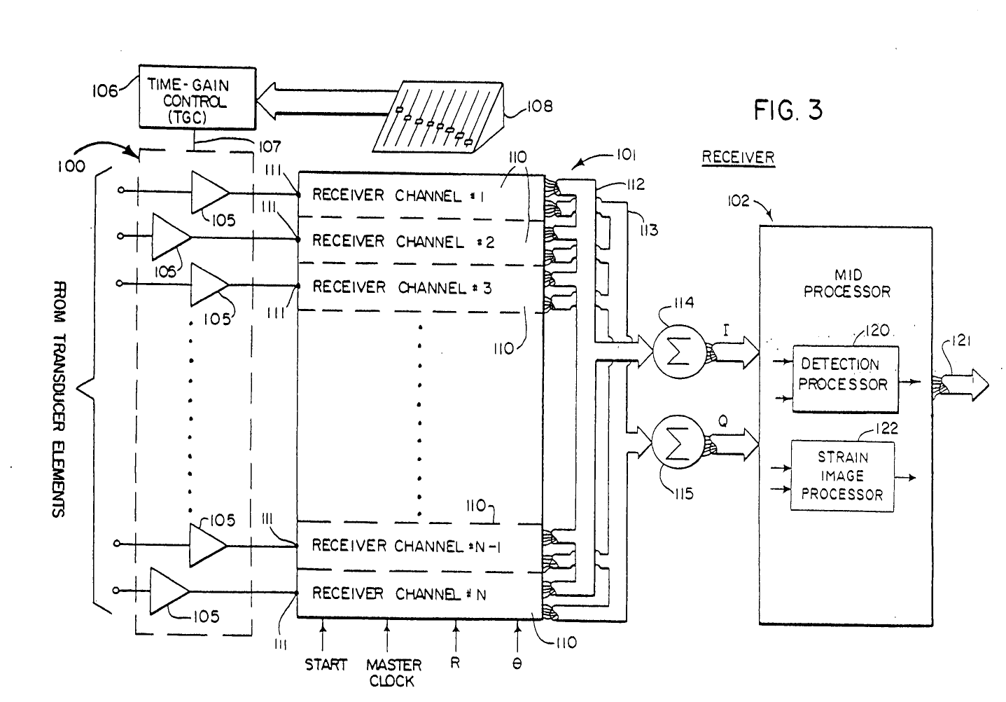

Referring particularly to FIG. 3, the receiver 14 is comprised of three sections: a time-gain control section 100, a beam forming section 101, and a mid processor 102. The time-gain control section 100 includes an amplifier 105 for each of the receiver channels and a time-gain control circuit 106. The input of each amplifier 105 is connected to a respective one of the transducer elements 12 to receive and amplify the echo signal which it receives. The amount of amplification provided by the amplifiers 105 is controlled through a control line 107 that is driven by the time-gain control circuit 106. As is well known in the art, as the range of the echo signal increases, its amplitude is diminished. As a result, unless the echo signal emanating from more distant reflectors is amplified more than the echo signal from nearby reflectors, the brightness of the image diminishes rapidly as a function of range (R). This amplification is controlled by the operator who manually sets TGC linear potentiometers 108 to values which provide a relatively uniform brightness over the entire range of the sector scan. The time interval over which the echo signal is acquired determines the range from which it emanates, and this time interval is divided into eight segments by the TGC control circuit 106. The settings of the eight potentiometers are employed to set the gain of the amplifiers 105 during each of the eight respective time intervals so that the echo signal is amplified in ever increasing amounts over the acquisition time interval.

The beam forming section 101 of the receiver 14 includes separate receiver channels 110. Each receiver channel 110 receives the analog echo signal from one of the TGC amplifiers 105 at an input 111, and it produces a stream of digitized output values on an I bus 112 and a Q bus 113. Each of these I and Q values represents a sample of the echo signal at a specific range (R). These samples have been delayed in the manner described above such that when they are summed at summing points 114 and 115 with the I and Q samples from each of the other receiver channels 110, they indicate the magnitude and phase of the echo signal reflected from a point P located at range R along the beam direction.

Referring still to FIG. 3, the mid processor section 102 receives the beam samples from the summing points 114 and 115. The I and Q values of each beam sample is a digital number which represents the in-phase and quadrature components of the reflected sound from a point. The mid processor 102 can perform a variety of calculations on these beam samples, where choice is determined by the type of image to be reconstructed. For example, if a conventional magnitude image is to be produced, a detection process indicated at 120 is implemented in which a digital magnitude M is calculated from each beam sample and output at 121.
M=\sqrt{I²+Q²}

The receiver 14 generates a stream of digital numbers at its output 121 which is applied to the input of the display system 17. As described above, this “scan data” can be used to produce an image indicative of the echo signal magnitude at locations in the region of interest being scanned.

To practice the present invention the mid processor 102 includes a strain image processor 122. In general ultrasound strain images are produced by comparing ultrasound echo data prior to and after a slight compression of the breast, to determine the tissue displacement at each location in the breast as a result of the compression. Tissue displacement is measured by tracking the movement of speckle patterns in many small tissue regions in the ultrasound echo data acquired from two successive frames obtained before and after the compression. Motion tracking is accomplished using cross-correlation or similar techniques. Strain is computed as the rate of change (or gradient) in the axial tissue displacement as a function of depth. The strain images are produced when the relative differences in tissue motion at each location in the breast are calculated and output at 121 to the display system 17. Harder areas (less tissue deformation with compression) of the breast appear darker on the strain image and softer areas (more tissue deformation with compression) appear brighter.

There are several methods known to those skilled in the art for producing ultrasound strain images. The preferred embodiment of the invention employs a method described in U.S. Pat. No. 6,508,768 which is incorporated herein by reference, and in a publication by Yanning Zhu et al “A Modified Blocking Matching Method For Real-Time Freehand Strain Imaging” Ultrasound Imaging 24, 161-176 (2002). The strain images are reconstructed from the beam samples at summing points 114 and 115 using a two-dimensional block-matching algorithm based on the sum-square difference method to estimate tissue displacement, and a linear regression method is used to estimate gradient, and thus strain, from the displacement field. The sizes of the kernel and search region used for displacement estimation are both approximately ½ the area of the ultrasound point spread function. Typically, frame-to-frame strain values are on the order of 1%, but are variable due to the freehand nature of the acquisition. Computed strain images are processed just prior to display in order to maintain a uniform average displayed brightness. To reduce the computational load and increase the real-time frame rate, strain data is computed only within an operator-specified strain region-of-interest.

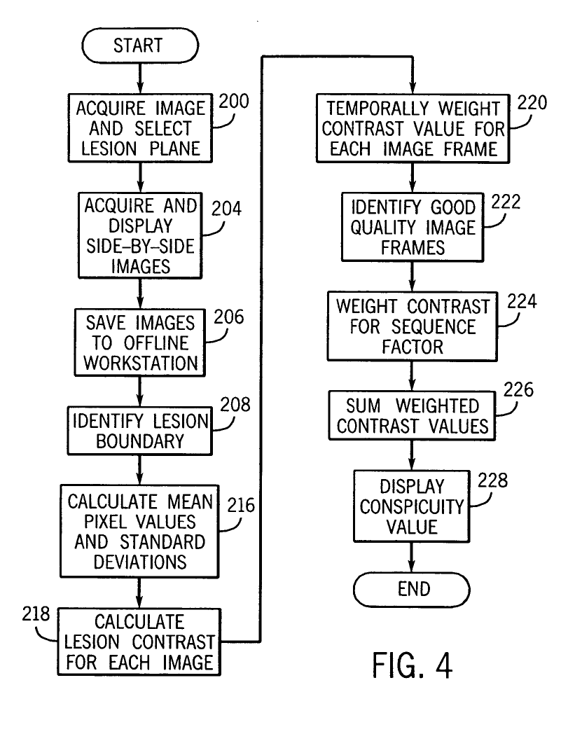

Referring particularly to FIG. 4 the first step in the protocol is to acquire B-mode images with the above described system and identity a scan plane that includes the lesion of concern as indicated at process block 200. The data acquisition and real-time display step is then begun as indicated at process block 204. As described above, approximately 100 frames of side-by-side B-mode and strain images are produced and displayed while the sonographer gently applies a varying axial force on the subject breast with the handheld acoustic transducer 11 as shown in FIG. 2. During this portion of the procedure the sonographer will make a visual assessment of the series of strain images as will be discussed in more detail below.

It is possible to distinguish benign from malignant breast lesions by visual assessment of the entire strain image sequence at the time of image acquisition. Observed characteristics such as “ease” of strain image acquisition and clear presentation of the lesion throughout the sequence correlate well with malignant lesions. Ease of strain imaging refers to the ability of the technologist to produce strain images of good quality very soon after beginning the strain imaging process, as well as the ability to produce many good-quality strain images during the acquisition period. This overall impression of ease of imaging and good lesion visibility is dubbed “conspicuity”. Lesions visually judged to be more conspicuous during acquisition are predicted to be malignant, while those judged as less conspicuous are predicted as benign. The criteria used for visual assessment of conspicuity are as follows:

1. Primary criteria: Overall visibility of the lesion in the strain lesion throughout the entire image sequence. Greater visibility suggests greater likelihood of malignancy.

2. Good early visibility of the lesion. Presence of high quality images that appear early in the sequence suggest greater likelihood of malignancy.

3. Good lesion contrast. Lesions that appear quite dark, with good contrast compared to the surrounding normal tissue, suggest greater likelihood of malignancy.

4. Sequences of images clearly showing the lesion. Lesions that are well-visualized in several images in a row suggest greater likelihood of malignance. Benign lesions tend to be well-visualized only in a few images sprinkled throughout the sequence.

5. Homogeneous appearance of the lesion. Lesions with a homogeneous dark appearance suggest greater likelihood of malignancy. Benign lesions tend to have a more heterogeneous, mixed appearance, e.g., a softer middle surrounded by a stiffer tissue.

6. Comparison of lesion size between B-mode and strain images. Lesions appearing visibly larger on the strain images than on the B-mode images suggest greater likelihood of malignancy. Most benign lesions tend to appear the same size in both images.

As indicated at process block 206, the acquired image frames (i.e. I and Q data) are stored in an offline processor for further processing according to the present invention. The offline processor is typically a stand-alone workstation that is networked with the ultrasound system, although the images may also be saved to a portable storage media and transported to the workstation. It is contemplated that future embodiments will include the functions performed by the offline processor as an integral part of the ultrasonic system.

Whereas the sonographer provides a visual assessment as to whether the lesion is benign or malignant based on an examination of the time series of B-mode and strain images, the workstation is programmed to provide a computer-aided diagnosis (CAD). As will now be described, the process used to provide a CAD metric takes advantage of several of the observational features listed above for the visual assessment. These features are translated to metrics that can be extracted from the image sequence, and a weighted sum of these metrics results in a single number that estimates the conspicuity of the lesion throughout the entire sequence.

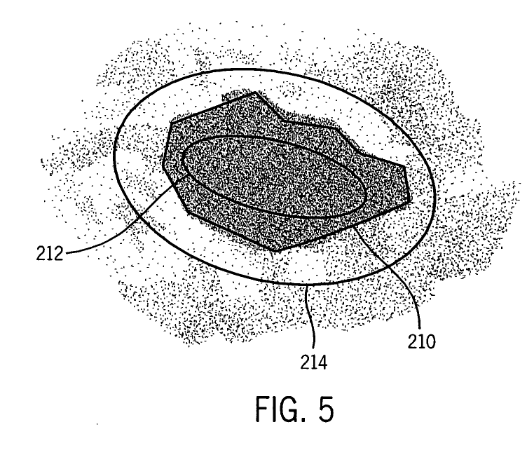

The strain images are reconstructed on the offline processor and the first step is to view one image from the strain sequence and use a computer mouse to roughly identify the lesion boundaries as indicated at process block 208. As shown in FIG. 5, the boundary of the lesion is marked manually as indicated by dotted line 210 and then a lesion ROI is computed as an elliptical area 212 located inside this boundary 210. The ellipse that defines the lesion is set to 75% the size of an ellipse fit to the manually marked boundary 210. A normal tissue ROI is then indicated by an elliptical outer annulus 214. This is determined by the elliptical-shaped annular space around the manually marked boundary 210 that has the same area as the lesion ROI 212 and is spaced outwardly from the manually marked boundary 210 approximately the same distance the lesion ROI 212 is spaced inward therefrom.

As indicated at process block 216 in FIG. 4, the mean strain image pixel value and the standard deviation of pixel values are then calculated for lesion ROI 212 and the normal tissue ROI in each of the series of strain images. As indicated at process block 218, the lesion contrast is then calculated for each strain image frame in the sequence. This lesion contrast value is the difference between the mean pixel value in the normal tissue ROI minus the mean pixel value in the lesion ROI 212, divided by the mean pixel value in the lesion ROI 212. The lesion pixel average is used in the denominator of this contrast calculation in order to emphasize lesions which exhibit very low strain pixel values (i.e. indicate very stiff tissue).

One factor described by sonographers as important to visual lesion assessment is the “ease of imaging” the lesion throughout the strain image sequence. This aspect of visual conspicuity is modeled by temporally weighting the strain image contrast measurement of each image frame as a function of the image frame number as indicated at process block 220. A Gaussian weighting is selected, with frame #1 having a weight of 1.00, and frame #100 having a weight of 0.05. If a lesion is “easy to image”, it is expected to appear with good contrast early in the strain sequence, and in this case, large weighting factors are applied to these images. High contrast images that appear very late in the sequence, after the sonographer has gained experience scanning the lesion, count less due to the lower values of the temporal weighting factor.

Another factor expected to be important in visual assessment of lesion conspicuity is the number of sequential strain images which clearly demonstrate the lesion. Contiguous sequences of images that clearly demonstrate the lesion will contribute to an increased sense of conspicuity to an observer, as compared with good quality frames separated by low contrast or noisy image frames. Contiguous sequences of good quality image frames also suggest a lesion that is “easy to scan”, so the contrast in these sequences is preferentially weighted.

As indicated at process block 222 the first step in this “contiguous sequence” weighting is to identify the strain images in the sequence that exceed an image quality threshold. Simultaneous thresholds of lesion contrast and lesion signal-to-noise ratio (SNR) are used to identify individual images of “good quality”. Lesion SNR is defined as the difference in the mean pixel values in the lesion and normal tissue ROIs, divided by the quadrature sum of the pixel standard deviations in the two ROIs. A threshold of 40% of the maximum lesion contrast and lesion SNR must be met for a strain image frame to be considered of“good quality”.

As indicated at process block 224, the temporally weighted contrast values calculated previously in step 220 are further weighted with the sequential weighting factor. This is done by multiplying the contrast value for each strain image frame by the square root of the number of “good quality” images in sequence. That is, if the strain image frame is a member of a sequence of N good quality images, then its contrast value is multiplied by \sqrt{N}.

As indicated by process block 226, the conspicuity metric is then calculated by summing the weighted contrast values for the entire sequence of image frames. This value is displayed or printed at process block 228.

In summary the conspicuity metric is the weighted sum of the lesion contrast measured in each image over the entire sequence. Weighting includes applying preferential Gaussian weighting to contrast in images appearing earlier in the sequence, and applying preferential root-N weighting to contrast in contiguous sequences of high-quality images. The method models the technologists' impression of conspicuity, and some of the individual factors described as being important to that impression. Variations in the preferred model of the conspicuity metric include the choice of the Gaussian temporal weighting, and “standard deviation” (described by the desired weight for frame 100), the choice of joint thresholding of lesions contrast and SNR to define contiguous sequences of “good quality images”, 0.40 (40% ) of maximum as the particular threshold level for both of these factors, and the root-N functional weighting of the contrast in frames in these sequences. Exponential, and linear time weighting functions were tried, and joint threshold values of 0.1, 0.2, 0.3, . . . , 0.9 were tested, but a general exhaustive search of these variable has not been undertaken. The conspicuity metric is unitless. The detail calculation of the preferred conspicuity metric is described by the following equation:

$$C = {\sum }_{f = 1}^{n} {exp ( \frac{1 - f}{{f}_{100 , 5 \%}} )}^{2} {\sqrt{N}}_{f , run} \{ \frac{{P}_{f , norm} - {P}_{f , lesion}}{{P}_{f , lesion}} \}$$

where: C=conspicuity metric for the strain sequence;
f=strain image frame number with the initial frame in the sequence being “1”;
n=total number of frames in the strain image sequence;
f_{100,5}%=constant to set Gaussian weight for frame 100 equal to 0.05;
N_{f,run}=length of the run of high quality frames, of which frame f is a part
P_{f,norm}=mean pixel value in normal tissue ROI in frame f; and
P_{f,lesion}=mean pixel value in lesion ROI in frame f.

The method was applied to 29 subjects and the results are listed in Table 1. The subjects are ordered according to the magnitude of the computed conspicuity metric, where “B” indicates benign, “M” indicates malignant, and “I” indicates indeterminate.

TABLE 1

| Computed | Visual | Biopsy | Case |
|---|---|---|---|
| “Conspicuity” | Assessment | Results | Number |
| 6.3 | B | M | 101 |
| 9.1 | B | B | 105 |
| 11.2 | B | B | 79 |
| 22.3 | B | B | 92 |
| 33.0 | B | B | 96 |
| 33.5 | B | B | 89 |
| 34.8 | B | B | 109 |
| 53.5 | B | B | 110 |
| 54.9 | B | B | 87 |
| 59.4 | M | M | 116 |
| 79.0 | M | M | 80 |
| 89.4 | M | M | 117 |
| 93.0 | M | M | 91 |
| 106.0 | M | M | 86 |
| 111.3 | M | M | 84 |
| 125.4 | M | M | 90 |
| 136.9 | M | B | 82 |
| 140.6 | B | B | 85 |
| 143.5 | M | M | 114 |
| 152.6 | M | M | 115 |
| 160.2 | M | M | 104 |
| 173.6 | M | M | 88 |
| 185.8 | M | M | 100 |
| 219.6 | M | M | 112 |
| 226.7 | M | M | 99 |
| 239.9 | M | M | 103 |
| 305.9 | M | M | 97 |
| 460.5 | M | M | 113 |
| 462.2 | I | B | 107 |

In order to derive a prediction of status for a specific lesion from the calculated conspicuity metric, a threshold must be defined. Conspicuity values above the threshold are taken to predict malignancy and values below the threshold are taken to predict benignity. Table 2 shows the performance of the conspicuity metric compared to the visual assessment results and pathology, for two different ranges of conspicuity metric threshold value.

TABLE 2

| Threshold | Accuracy | Accuracy | | |
|---|---|---|---|---|
| Range | (Visual Assessment) | (Pathology) | Sensitivity | Specificity |
| 55-59 | 96.6% | 86.2% | 94.4% | 72.7% |
| 35-53 | 89.7% | 79.3% | 94.4% | 54.4% |

A threshold chosen in the narrow range of 55-59 results in the best performance of the conspicuity metric, while a threshold chosen in the wider range of 35-53 yields somewhat poorer performance. Thresholds in the 35-53 range result in cases 110 and 87 being incorrectly designated as malignant (false-positive), thus lowering specificity. Any threshold chosen in the range of 35-59 (a range of 25 possible values) yields performance at least as good as that designated in the “35-53” threshold range column in Table 2. The 54.4% specificity value suggests that at least 50% of biopsies that would have otherwise been ordered, ultimately resulting in benign findings, would be avoided through the use of the conspicuity metric alone.

This conspicuity measurement method falls generally into the category of computer-aided diagnosis. Like other common applications of CAD in radiology, we expect that this approach will be most useful when used in conjunction with visual interpretation of the strain image data. We expect that the method may also be improved by considering additional strain image features that contribute to overall lesion conspicuity (e.g., edge profile and lesion homogeneity), or that correlate with other known lesion characteristics. For example, the fact that benign lesions are mobile suggests measuring strain image decorrelation due to elevational lesion motion and lateral lesion displacement, and incorporating these into the method as additional weighting factors. In general, applying conventional approaches for developing and optimizing CAD classifiers may yield greater separation between benign and malignant lesion measurements.

1. A method for diagnosing a lesion in a subject, the steps comprising:
a) acquiring a series of strain images of the lesion and surrounding tissues using an ultrasound imaging system;
b) calculating a lesion contrast value for each strain image based on the mean pixel value of the lesion and the pixel value of surrounding tissues in the strain image;
c) weighting each lesion contrast value using a weighting factor derived from information in one or more of the strain images; and
d) producing a conspicuity metric by summing the weighted lesion contrast values, and wherein a diagnosis is made based in part on the value of this conspicuity metric.

2. The method as recited in claim 1 in which the lesion is in the subject's breast and step a) is performed by positioning an ultrasonic transducer on the breast and applying a variable axial force to the breast while the series of strain images are acquired.

3. The method as recited in claim 2 in which the variable axial force is applied by moving the ultrasonic transducer.

4. The method as recited in claim 1 in which step a) includes acquiring a corresponding series of B-mode images.

5. The method as recited in claim 1 in which the lesion contrast value calculation in step b) includes:
b)i) calculating the mean image pixel value in the lesion;
b)ii) calculating the mean image pixel value in tissues surrounding the lesion; and
b)iii) calculating the difference between the two calculated mean pixel values.

6. The method as recited in claim 5 in which step b) further includes:
b)iv) dividing the difference calculated in step b)iii) by the lesion mean pixel value calculated in step b)i).

7. The method as recited in claim 1 in which step c) includes:
c)i) calculating a weighting factor for each strain image which weights images acquired at the beginning of the series higher than images acquired at the end of the series.

8. The method as recited in claim 7 in which the first image in the series is weighted at substantially 1 and subsequent images in the series are Gaussian weighted.

9. The method as recited in claim 7 in which step c) also includes:
c)ii) calculating a sequence weighting factor for each strain image which weights according to the number of consecutive good quality images of which the strain image is a part.

10. The method as recited in claim 1 in which step c) includes calculating a contiguous sequence weighting factor for each strain image which weights according to the number of consecutive good quality images of which the strain image is a part.

11. The method as recited in claim 10 in which the contiguous sequence weighting factor is \sqrt{N}, where N is the number of consecutive good quality images.

12. The method as recited in claim 1 in which step d) is performed by making the calculation:
$$C = {\sum }_{f = 1}^{n} {exp ( \frac{1 - f}{{f}_{100 , 5 \%}} )}^{2} {\sqrt{N}}_{f , run} \{ \frac{{P}_{f , norm} - {P}_{f , lesion}}{{P}_{f , lesion}} \}$$
where: C=conspicuity metric for the strain sequence;
f=strain image frame number;
n=total number of frames in the strain image sequence;
f_{100,5}%=constant to set Gaussian weight for frame 100 equal to 0.05;
N_{f,run}=length of the run of high quality frames, of which frame f is a part
P_{f,norm}=mean pixel value in normal tissue ROI in frame f; and
P_{f,lesion}=mean pixel value in lesion ROI in frame f.

13. A method for non-invasively diagnosing a breast lesion, the steps comprising:
a) acquiring a series of strain images of the lesion and surrounding tissues using an ultrasound imaging system by:
a)i) positioning an ultrasound transducer on the breast; and
a)ii) applying a variable axial force to the breast while the series of strain images are acquired;
b) visually assessing the status of the lesion based on the observed conspicuity of the lesion in the acquired strain images;
c) calculating a conspicuity metric from the acquired strain images; and
d) making a diagnosis based on the visual assessment in step b) and the conspicuity metric calculated in step c).

14. The method as recited in claim 13 in which step c) includes:
c)i) calculating a lesion contrast value for each strain image;
c)ii) weighting each lesion contrast value using a weighting factor derived from information in a strain image;
c)iii) summing the weighted lesion contrast values to calculate the conspicuity metric.

15. The method as recited in claim 14 in which step c)i) is performed by:
identifying lesion pixels in each strain image;
identifying surrounding tissue pixels in each strain image;
calculating a mean pixel value of the identified lesion in each strain image;
calculating a mean pixel value of identified surrounding tissue pixels in each strain image; and
calculating the difference between the two calculated mean pixel values for each strain image.

16. The method as recited in claim 14 in which step c)i) further includes dividing the difference between the two calculated mean pixel values by the lesion mean pixel value.
